(19) United States
(12) Patent Application Publication
(10) Pub. No.: US 2018/0003725 A1
(43) Pub. Date: Jan. 4, 2018

(54) METHODS AND SYSTEMS FOR PREDICTING BLEEDING RISK AND DOSE OF PLASMINOGEN ACTIVATOR

(71) Applicants: Jeffrey KLINE, Carmel, IN (US); Indiana University Research and Technology Corporation, Indianapolis, IN (US)

(72) Inventor: Jeffrey Kline, Carmel, IN (US)

(73) Assignee: Indiana University Research and Technology Corporation, Indianapolis, IN (US)

(21) Appl. No.: 15/536,124

(22) PCT Filed: Jan. 21, 2016
(86) PCT No.: PCT/US16/14279

§ 371 Date: Jun. 14, 2017

Related U.S. Application Data
(60) Provisional application No. 62/106,436, filed on Jan. 22, 2015.

(51) Int. Cl. G01N 33/86; G06F 19/00

(52) U.S. Cl. CPC G01N 33/86; G06F 19/704; G01N 2800/224; G01N 2800/52; G06F 19/3431

(57) ABSTRACT

The present disclosure provides a method and system for estimating the clinical responsiveness of a patient to a dose of a plasminogen activating agent to treat a thrombosis, comprising determining a concentration of α2-antiplasmin in a blood sample of the patient, determining a concentration of activated fibrinolysis inhibitor (“TAFI”) in the blood sample, determining a concentration of plasminogen activator Inhibitor 1 (“PAI-1”) in the blood sample, computing a clot lysis time (“CLT”) based on the concentrations of a2-antiplasmin, TAFI and PAI-1 using the equation CLT=−2,813.6+31.1*a2-antiplasmin (percent activity)+31.1*TAFI (percent activity)+1.49 PAI-1 (ug/L), and determining that the patient is at increased risk of hemorrhage when the computed CLT is less than a first predetermined cutoff time.

### RELATED APPLICATIONS

The present application claims priority to U.S. Provisional Application Ser. 62/106,436, filed Jan. 22, 2015, and entitled “METHODS AND SYSTEMS FOR PREDICTING BLEEDING RISK AND DOSE OF PLASMINOGEN ACTIVATOR,” the complete disclosure of which is expressly incorporated herein by reference.

### TECHNICAL FIELD

The present disclosure relates to computer-based methods and systems for predicting the magnitude of plasmatic resistance to plasminogen activator-mediated fibrinolysis in-vivo.

### BACKGROUND

Acute vascular thrombosis (including: coronary, cerebrovascular, and pulmonary thrombosis) causes more deaths than any other disease process in western society (S. L. Murphy, J. Xu, K. D. Kochanek. Deaths: Final data for 2010. National Vital Statistics Reports: From the Centers for Disease Control and Prevention, National Center for Health Statistics, National Vital Statistics System 61 (2013) 1-117). Blood vessels obstructed by thrombus can be recannulized within hours by enzymatic digestion, mechanical disruption (e.g., angioplasty or catheter disruption) or by a combination of the two methods. In current clinical practice, enzymatic digestion of clots is accomplished by administering an enzyme such as recombinant tissue plasminogen activator (“rt-PA”), tenecteplase, retaplase, urokinase or streptokinase to activate circulating and clot-bound plasminogen to plasmin (K. Ouriel, E. L. Welch, C. K. Shorten, K. Geary, W. M. Fiore, C. Cimino. Comparison of streptokinase, urokinase, and recombinant tissue-plasminogen activator in an in-vitro model of venous thrombolysis, J. Vasc. Surg. 22 (1995) 593-597; R. Fears, M. J. Hibbs, R. A. G. Smith. Kinetic-studies on the interaction of streptokinase and other plasminogen activators with plasminogen and fibrin, Biochem. J. 229 (1985) 555-558). Activated plasmin, in turn, cleaves cross-linked γ-chains in the D-domain of fibrin (Aα 148-160) to effectively digest the thrombus (L. Medved, W. Nieuwenhuizen. Molecular mechanisms of initiation of fibrinolysis by fibrin, Thromb. Haemost. 89 (2003) 409-419). Plasminogen is a 91 kDa zymogen containing 791 amino acids, produced in-vivo by the liver, and is heavily glycosylated (2% carbohydrate) in its circulating form (FIG. 1) (R. H. P. Law, T. Caradoc-Davies, N. Cowieson, A. J. Horvath, A. J. Quek, J. A. Encarnacao, D. Steer, A. Cowan, Q. Zhang, B. G. C. Lu, R. N. Pike, A. I. Smith, P. B. Coughlin, J. C. Whisstock. The X-ray crystal structure of full-length human plasminogen, Cell Reports 1 (2012) 185-190). When cleaved at Arg561-Val562, plasminogen produces plasmin, a serine protease with a trypsin-like active site (Supporting Information Figure S1). Plasmin binds to thrombi via electrostatic attraction between its five kringle (K) domains to the exposed lysine residues on fibrin with a Kd=0.5 μM for lys-plasmin and Kd=5 μM for glu-plasmin (M. A. Lucas, L. J. Fretto, P. A. Mckee. The binding of human-plasminogen to fibrin and fibrinogen, J. Biol. Chem. 258 (1983) 4249-4256.). In order, K4 has the least, K1-K3 have moderate, and K5 has the highest affinity fibrin binding (V. V. Novokhatny, Y. V. Matsuka, S. A. Kudinov. Analysis of ligand-binding to kringles-4 and kringles-5 fragments from human-plasminogen, Thromb. Res. 53 (1989) 243-252; S. G. Mccance, N. Menhart, F. J. Castellino. Amino-acid-residues of the kringle-4 and kringle-5 domains of human plasminogen that stabilize their interactions with omega-amino acid ligands, J. Biol. Chem. 269 (1994) 32,405-32,410). Plasmin's activity is rapidly neutralized in plasma by the circulating proteins α2-antiplasmin, C1-inhibitor, and macroglobulin (P. K. Anonick, B. B. Wolf, S. L. Gonias. Regulation of plasmin, miniplasmin, and streptokinase-plasmin complex by alpha-2-antiplasmin, alpha-2-macroglobulin, and antithrombin-iii in the presence of heparin, Thromb. Res. 59 (1990) 449-462; H. S. Cummings, F. J. Castellino. Interaction of human plasmin with human alpha-2-macroglobulin, Biochemistry (N.Y.) 23 (1984) 105-111). The serpin α2-antiplasmin provides the most rapid and avid inhibition, whereby an Arg-Met residue binds directly to the serine residue in plasmin's active site with a rate constant of 4×107 M-1Sec-1 (P. K. Anonick, B. B. Wolf, S. L. Gonias. Regulation of plasmin, miniplasmin, and streptokinase-plasmin complex by alpha-2-antiplasmin, alpha-2-macroglobulin, and antithrombin-iii in the presence of heparin, Thromb. Res. 59 (1990) 449-462).

Lack of target specificity poses the largest threat to the clinical therapeutic index of the plasminogen activators. Even when rt-PA is infused directly via a catheter buried within the thrombus, some degree of systemic plasminogen activation occurs, fibrinogenolysis and increased bleeding risk. Disruption of fibrin-based thrombi in the cerebral microvasculature that normally serve to prevent perfusion into vessels deteriorated by mechanisms such as hyaline degeneration, (lipohyalinosis), leading to the dreaded complication of intracerebral hemorrhage. Intracerebral bleeding occurs in approximately 1-2% of patients treated with systemic full-dose fibrinolysis (e.g., 0.5-0.9 mg/kg of tissue plasminogen activator) for acute pulmonary embolism (“PE”) and in approximately 5-10% of patients with ischemic stroke (Chatterjee S, Chakraborty A, Weinberg I et al. Thrombolysis for pulmonary embolism and risk of all-cause mortality, major bleeding, and intracranial hemorrhage: a meta-analysis. JAMA. 2014; 311(23):2414-2421; Mullen M T, Pisapia J M, Tilwa S et al. Systematic review of outcome after ischemic stroke due to anterior circulation occlusion treated with intravenous, intra-arterial, or combined intravenous+intra-arterial thrombolysis. Stroke. 2012; 43 (9): 2350-2355).

Recent work has demonstrated efficacy with half-dose tPA in reducing right ventricular dysfunction after PE compared with no fibrinolysis, while potentially decreasing the bleeding risk associated with full-dose tPA (Zhang Z, Zhai Z G, Liang L R et al. Lower dosage of recombinant tissue-type plasminogen activator (rt-PA) in the treatment of acute pulmonary embolism: a systematic review and meta-analysis. Thromb Res. 2014; 133(3):357-363). Similarly, lower doses of fibrinolytic agents for ischemic stroke have been associated with significantly lower rates of intracranial hemorrhage (Mullen M T, Pisapia J M, Tilwa S et al. Systematic review of outcome after ischemic stroke due to anterior circulation occlusion treated with intravenous, intra-arterial, or combined intravenous+intra-arterial thrombolysis. Stroke. 2012; 43(9):2350-2355; Wardlaw J M, Koumellis P, Liu M. Thrombolysis (different doses, routes of administration and agents) for acute ischaemic stroke. Cochrane Database Syst Rev. 2013; 5:CD000514).

The primary regulatory proteins that restrain the action of plasminogen activators include plasminogen activator inhibitor-1 (“PAI-1”), that circulates with a plasma concentration of approximately 0.01 ug/L. PAI-1 is a serpin protein that rapidly and stoichiometrically inhibits plasminogen activators by destroying the active site. Similarly, α₂-antiplasmin acts to neutralize activated free plasmin in a rapid and specific manner both in plasma phase and on the fibrin surface. Thrombin-activating fibrinolysis inhibitor (“TAFI”) is a glycoprotein that impairs fibrinolysis by removing carboxyterminal arginine and lysine residues that attract the kringles of plasmin. Taken together, all three proteins exert an inhibitory effect on fibrinolysis (Marder V J, Francis C W. Physiological regulation of fibrinolysis. In: Colman R W, Marder V J, Clowes A W et al., eds. Hemostasis and Thrombosis. Basic prinicples and clinical practice. 5 ed. Philadelphia: Lippincott and Williams; 2006). Elevated concentrations of these proteins can be hypothesized to decreased or incomplete lysis effect of fibrinolytic treatment, and lowered concentrations of these proteins can be hypothesized to increased risk of hemorrhage from standard dose plasminogen activating agents.

Prior data are known that increase risk of hemorrhage in association with therapeutic use of plasminogen inhibitors, including age, comorbid conditions (prior stroke, cancer, renal failure, liver disease, diabetes mellitus, atrial fibrillation, congestive heart failure), recent trauma, elevated blood pressure, and size of stroke (Whiteley W N, Slot K B, Fernandes P et al. Risk factors for intracranial hemorrhage in acute ischemic stroke patients treated with recombinant tissue plasminogen activator: a systematic review and meta-analysis of 55 studies. Stroke. 2012; 43(11):2904-2909; Flint A C, Gupta R, Smith W S et al. The THRIVE score predicts symptomatic intracerebral hemorrhage after intravenous tPA administration in SITS-MOST Int J Stroke. 2014; 9(6):705-710). Biomarkers that have been studied for prediction of intracerebral hemorrhage after stroke treatment with plasminogen activator therapy include fibronectin and matrix metalloproteinase-9 levels (Castellanos M, Sobrino T, Millan M et al. Serum cellular fibronectin and matrix metalloproteinase-9 as screening biomarkers for the prediction of parenchymal hematoma after thrombolytic therapy in acute ischemic stroke: a multicenter confirmatory study. Stroke. 2007; 38(6):1855-1859). No studies, however, have reported a scoring system to predict risk of hemorrhage or incomplete lysis associated with plasminogen activation in pulmonary embolism and no scoring system for prediction of clinical response to fibrinolytic treatment has incorporated the biomarkers PAI-1, α₂-antiplasmin or TAFI.

It is known that patients with metabolic syndrome (obesity, elevated blood lipids and insulin resistance) and diabetes mellitus both have intrinsic resistance to fibrinolysis, or the so-called hypofibrinolytic condition (Alessi M C, Juhan-Vague I. Metabolic syndrome, haemostasis and thrombosis. Thromb Haemost. 2008; 99(6):995-1000; Dunn E J, Philippou H, Ariens R A et al. Molecular mechanisms involved in the resistance of fibrin to clot lysis by plasmin in subjects with type 2 diabetes mellitus. Diabetologia. 2006; 49(5): 1071-1080). Accordingly, patients with these conditions serve as logical control patients to compare results with patients who have thrombosis.

### SUMMARY

In one embodiment, the present disclosure provides a method for estimating the clinical responsiveness of a patient to a dose of a plasminogen activating agent to treat a thrombosis, comprising determining, using at least one biomarker measurement system, a concentration of α2-antiplasmin in a blood sample of the patient, determining, using the at least one biomarker measurement system, a concentration of activated fibrinolysis inhibitor (“TAFI”) in the blood sample, determining, using the at least one biomarker measurement system, a concentration of plasminogen activator Inhibitor 1 (“PAI-1”) in the blood sample, computing, using a computing device, a clot lysis time (“CLT”) based on the concentrations of α2-antiplasmin, TAFI and PAI-1 using the equation CLT=−2,813.6+31.1*α2-antiplasmin (percent activity)+31.1*TAFI (percent activity)+1.49 PAI-1 (ug/L), and determining, using the computing device, that the patient is at increased risk of hemorrhage when the computed CLT is less than a first predetermined cutoff time.

As will be appreciated by the skilled person, in embodiments the method allows for the analysis of a patient blood sample using a computing device that is external to the patient. In embodiments, the method is thus an in vitro method for estimating the clinical responsiveness of a patient. In such an in vitro method, the determining of concentrations of α2-antiplasmin, activated fibrinolysis inhibitor (“TAFT”) and/or plasminogen activator Inhibitor 1 (“PAI-1”) take place outside of the body of a patient. For example, the concentration of the markers described above can be determined using various testing protocols in a laboratory, as described more fully below.

In one embodiment, the first predetermined cutoff time is approximately 4,926 seconds. In another embodiment, the method further includes determining, using the computing device, that the patient is at an increased risk of clinical failure with treatment with the plasminogen activating agent when the computed CLT is greater than a second predetermined cutoff time, the second predetermined cutoff time being greater than the first predetermined cutoff time. The second predetermined cutoff time may be approximately 15,247 seconds. In embodiments, the concentrations of α2-antiplasmin and TAFI are percentages of a normative value. In embodiments, the first cutoff time corresponds to the mean CLT value of healthy patients that show no increased hemorrhage risk. In other words, the method is thus able to determine increased risk of hemorrhage in a patient, relative to a patient that demonstrates a normal or lower risk of hemorrhage.

In embodiments, the at least one biomarker measurement system includes a chromogenic assay for determining the concentration of α2-antiplasmin, TAFI and PAI-1 in the blood sample.

In embodiments, the method further includes responding to the computed CLT being less than the first predetermined cutoff time by providing a reduced dose plasminogen activator fibrinolytic treatment to the patient.

Optionally, the method may include providing a blood sample from a patient.

As will be appreciated, the method of the present invention may be used as a screening method for identifying, within a population, one or more patients with an increased risk of hemorrhage, or increased risk of clinical failure, when dosed with a plasminogen activating agent to treat a thrombosis. The invention may thus provide a screening method for identifying, within a population, one or more patients with an increased risk of hemorrhage when dosed with a plasminogen activating agent to treat a thrombosis, the method comprising:

a) determining, using at least one biomarker measurement system, the concentration of α2-antiplasmin, activated fibrinolysis inhibitor (“TAFI”) and plasminogen activator Inhibitor 1 (“PAI-1”) in a blood sample from each patient;

b) computing, using a computing device, a clot lysis time (“CLT”) based on the concentrations of α2-antiplasmin, TAFI and PAI-1 using the equation CLT=−2,813.6+31.1*α2-antiplasmin (percent activity)+31.1*TAFI (percent activity)+1.49 PAI-1 (ug/L); and

c) determining, using the computing device, that a patient is at increased risk of hemorrhage when the computed CLT is less than a first predetermined cutoff time, or determining, using the computing device, that a patient is at increased risk of clinical failure when the computed CLT is greater than a second predetermined cutoff time, the second predetermined cutoff time being greater than the first predetermined cutoff time.

In another embodiment, the present disclosure provides a system for estimating the clinical responsiveness of a patient to administration of a plasminogen activating agent to treat a thrombosis, comprising at least one biomarker measurement system for determining a concentration of α2-antiplasmin in a blood sample of the patient, a concentration of activated fibrinolysis inhibitor (“TAFI”) in the blood sample, and a concentration of plasminogen activator Inhibitor 1 (“PAI-1”) in the blood sample, a computing device including a processor and a memory including instructions which when executed by the processor cause the computing device to compute a clot lysis time (“CLT”) based on the concentrations of α2-antiplasmin, TAFI and PAI-1 using the equation CLT=−2,813.6+31.1*α2-antiplasmin (percent activity)+31.1*TAFI (percent activity)+1.49 PAI-1 (ug/L), and a user interface configured to provide a user of the computing device information that the patient is at increased risk of hemorrhage when the computed CLT is less than a first predetermined cutoff time.

The computing device may be in communication with the at least one biomarker measurement system. In embodiments, execution of the instructions by the processor may also cause the computing device to compare the computed CLT to a first predetermined cutoff time.

In embodiments, the first predetermined cutoff time is approximately 4,926 seconds. The user interface may be further configured to provide the user information that the patient is at an increased risk of clinical failure with treatment with the plasminogen activating agent when the computed CLT is greater than a second predetermined cutoff time, the second predetermined cutoff time being greater than the first predetermined cutoff time.

In embodiments, the second predetermined cutoff time is approximately 15,247 seconds.

In embodiments, the concentrations of α2-antiplasmin and TAFI are percentages of a normative value.

In embodiments, the at least one biomarker measurement system includes a chromogenic assay for determining the concentration of α2-antiplasmin, TAFI and PAI-1 in the blood sample.

In a further aspect of the invention there is provided a panel of biomarkers for estimating the clinical responsiveness of a patient to a dose of a plasminogen activating agent, the biomarkers comprising, consisting essentially of, or consisting of α2-antiplasmin, TAFI and PAI-1.

There is also provided the use of α2-antiplasmin, TAFI and PAI-1 as biomarkers for estimating the clinical responsiveness of a patient to a dose of a plasminogen activating agent to treat a thrombosis. For example, the concentrations of these biomarkers in a blood sample of the patient may be used to estimate the clinical responsiveness of a patient to a dose of a plasminogen activating agent. In embodiments, the use comprises determining, using at least one biomarker measurement system, a concentration of α2-antiplasmin in a blood sample of the patient, determining, using the at least one biomarker measurement system, a concentration of activated fibrinolysis inhibitor (“TAFI”) in the blood sample, and determining, using the at least one biomarker measurement system, a concentration of plasminogen activator Inhibitor 1 (“PAI-1”) in the blood sample. The use may optionally further comprise calculating a clot lysis time based on the concentrations of α2-antiplasmin, TAFI and PAI-1 using the equation CLT=−2,813.6+31.1*α2-antiplasmin (percent activity)+31.1*TAFI (percent activity)+1.49 PAI-1 (ug/L), and determining, that the patient is at increased risk of hemorrhage when the calculated CLT is less than a first predetermined cutoff time, or determining, that a patient is at increased risk of clinical failure when the calculated CLT is greater than a second predetermined cutoff time, the second predetermined cutoff time being greater than the first predetermined cutoff time. The calculating may optionally be computing, using a computing device, a clot lysis time (“CLT”) based on the concentrations of α2-antiplasmin, TAFI and PAI-1 using the equation CLT=−2,813.6+31.1*α2-antiplasmin (percent activity)+31.1*TAFI (percent activity)+1.49 PAI-1 (ug/L), and determining, using the computing device, that the patient is at increased risk of hemorrhage when the computed CLT is less than a first predetermined cutoff time, or determining, using the computing device, that a patient is at increased risk of clinical failure when the computed CLT is greater than a second predetermined cutoff time, the second predetermined cutoff time being greater than the first predetermined cutoff time.

There is also provided a kit for estimating the clinical responsiveness of a patient to a dose of a plasminogen activating agent to treat a thrombosis, the kit comprising a biomarker measurement assay capable of determining the concentration of α2-antiplasmin, activated fibrinolysis inhibitor (“TAFI”) and plasminogen activator Inhibitor 1 (“PAI-1”) in a blood sample of a patient. In embodiments, the kit may further comprise a computing device including a processor and a memory including instructions, which, when executed by the processor, cause the computing device to compute a clot lysis time (“CLT”) based on the concentrations of α2-antiplasmin, TAFI and PAI-1 using the equation CLT=−2,813.6+31.1*α2-antiplasmin (percent activity)+31.1*TAFI (percent activity)+1.49 PAI-1 (ug/L), and a user interface configured to provide a user of the computing device information that the patient is at increased risk of hemorrhage when the computed CLT is less than a first predetermined cutoff time, or determining, using the computing device, that a patient is at increased risk of clinical failure when the computed CLT is greater than a second predetermined cutoff time, the second predetermined cutoff time being greater than the first predetermined cutoff time. In embodiments, the kit may comprise a container for a blood sample collected from a patient. In further embodiments the kit may further comprise instructions for measuring the concentration of each biomarker and determining the probability that the patient is at increased risk of hemorrhage when the computed CLT is less than a first predetermined cutoff time. Typically, the biomarker measurement assay comprises detection molecules that specifically bind to α2-antiplasmin, TAFI and/or PAI-1, such as antibodies, antibody fragments, or nucleic acids. Such molecules can be used in the methods described herein. For example, such molecules may form part of a biomarker measurement system as described above.

There is also provided a biomarker measurement assay for estimating the clinical responsiveness of a patient to a dose of a plasminogen activating agent to treat a thrombosis, the assay being capable of determining the concentration of α2-antiplasmin, activated fibrinolysis inhibitor (“TAFI”) and plasminogen activator Inhibitor 1 (“PAI-1”) in a blood sample of a patient. In embodiments, the assay is a chromogenic assay. Typically, the biomarker measurement assay comprises detection molecules that specifically bind to α2-antiplasmin, TAFI and/or PAI-1, such as antibodies, antibody fragments, or nucleic acids. Such molecules can be used in the methods described herein. For example, such molecules may form part of a biomarker measurement system as described above.

In embodiments, the biomarker measurement assay is a biomarker measurement composition for estimating the clinical responsiveness of a patient to a dose of a plasminogen activating agent to treat a thrombosis, the composition being capable of determining the concentration of α2-antiplasmin, activated fibrinolysis inhibitor (“TAFI”) and plasminogen activator Inhibitor 1 (“PAI-1”) in a blood sample of a patient and further comprising pharmaceutically acceptable excipients. In embodiments, the composition is a chromogenic assay. Typically, the biomarker measurement composition comprises detection molecules that specifically bind to α2-antiplasmin, TAFI and/or PAI-1, such as antibodies, antibody fragments, or nucleic acids. Such molecules can be used in the methods described herein. For example, such molecules may form part of a biomarker measurement system as described above. In embodiments, the invention provides the use of a biomarker measurement composition in a method of estimating the use of clinical responsiveness of a patient to a dose of a plasminogen activating agent to treat a thrombosis, the method comprising determining, using at least one biomarker measurement system comprising the biomarker measurement composition, a concentration of α2-antiplasmin in a blood sample of the patient, determining, using the at least one biomarker measurement system, a concentration of activated fibrinolysis inhibitor (“TAFI”) in the blood sample, determining, using the at least one biomarker measurement system, a concentration of plasminogen activator Inhibitor 1 (“PAI-1”) in the blood sample, calculating a clot lysis time (“CLT”) based on the concentrations of α2-antiplasmin, TAFI and PAI-1 using the equation CLT=−2,813.6+31.1*α2-antiplasmin (percent activity)+31.1*TAFI (percent activity)+1.49 PAI-1 (ug/L), and determining that the patient is at increased risk of hemorrhage when the calculated CLT is less than a first predetermined cutoff time, or determining that a patient is at increased risk of clinical failure when the calculated CLT is greater than a second predetermined cutoff time, the second predetermined cutoff time being greater than the first predetermined cutoff time. The calculating may optionally be computing, using a computing device, a clot lysis time (“CLT”) based on the concentrations of α2-antiplasmin, TAFI and PAI-1 using the equation CLT=−2,813.6+31.1*α2-antiplasmin (percent activity)+31.1*TAFI (percent activity)+1.49 PAI-1 (ug/L), and determining, using the computing device, that the patient is at increased risk of hemorrhage when the computed CLT is less than a first predetermined cutoff time, or determining, using the computing device, that a patient is at increased risk of clinical failure when the computed CLT is greater than a second predetermined cutoff time, the second predetermined cutoff time being greater than the first predetermined cutoff time.

There is also provided a biomarker measurement assay for use in a method of treating thrombosis in a patient, the biomarker measurement assay being capable of determining the concentration of α2-antiplasmin, activated fibrinolysis inhibitor (“TAFI”) and plasminogen activator Inhibitor 1 (“PAI-1”) in a blood sample of a patient, comprising:

a) estimating the clinical responsiveness of the patient to a dose of a plasminogen activating agent to treat a thrombosis, comprising determining, using at least one biomarker measurement system comprising the biomarker measurement assay, a concentration of α2-antiplasmin in a blood sample of the patient, determining, using the at least one biomarker measurement system, a concentration of activated fibrinolysis inhibitor (“TAFI”) in the blood sample, determining, using the at least one biomarker measurement system, a concentration of plasminogen activator Inhibitor 1 (“PAI-1”) in the blood sample, computing, using a computing device, a clot lysis time (“CLT”) based on the concentrations of α2-antiplasmin, TAFI and PAI-1 using the equation CLT=−2,813.6+31.1*α2-antiplasmin (percent activity)+31.1*TAFI (percent activity)+1.49 PAI-1 (ug/L),

b) determining, using the computing device, that
i) the patient is at increased risk of hemorrhage when the computed CLT is less than a first predetermined cutoff time; or
ii) the patient is not at increased risk of hemorrhage when the computed CLT is not less than a first predetermined cutoff time; and

c) if the patient is not determined as being at increased risk of hemorrhage, administering to the patient a dose of a plasminogen activating agent suitable to treat a thrombosis, or administering adjunctive treatments, such as increased or prolonged or repeated dosing of plasminogen activating agent; or

d) if the patient is determined as being at increased risk of hemorrhage, then
i) administering to the patient a reduced dose of a plasminogen activating agent, relative to the regular dose to be administered to a patient that is not determined as being at increased risk of hemorrhage, or
ii) treating the patient with one or more anti-thrombotic agents that are not plasminogen activating agents, such as an agent selected from the group consisting of direct fibrinolytic agents, plasmin, delta plasmin, miniplasmin, microplasmin, lumbrokinase and nattokinase.

In embodiments, the assay is a chromogenic assay. Typically, the biomarker measurement assay comprises detection molecules that specifically bind to α2-antiplasmin, TAFI and/or PAI-1, such as antibodies, antibody fragments, or nucleic acids. In embodiments, the biomarker measurement assay is a biomarker measurement composition for estimating the clinical responsiveness of a patient to a dose of a plasminogen activating agent to treat a thrombosis, the composition being capable of determining the concentration of α2-antiplasmin, activated fibrinolysis inhibitor (“TAFI”) and plasminogen activator Inhibitor 1 (“PAI-1”) in a blood sample of a patient and further comprising pharmaceutically acceptable excipients. Typically, the biomarker measurement composition comprises detection molecules that specifically bind to α2-antiplasmin, TAFI and/or PAI-1, such as antibodies, antibody fragments, or nucleic acids. Such molecules can be used in the methods described herein.

There is also provided a plasminogen activating agent for use in a method of treating thrombosis in a patient, wherein the patient has been determined as being suitable for treatment with the plasminogen activating agent when a clot lysis time (CLT) is less than a first predetermined cutoff time, the method comprising determining, using at least one biomarker measurement system, a concentration of α2-antiplasmin in a blood sample of the patient, determining, using the at least one biomarker measurement system, a concentration of activated fibrinolysis inhibitor (“TAFI”) in the blood sample, and determining, using the at least one biomarker measurement system, a concentration of plasminogen activator Inhibitor 1 (“PAI-1”) in the blood sample, wherein the clot lysis time is calculated based on the concentrations of α2-antiplasmin, TAFI and PAI-1 using the equation CLT=−2,813.6+31.1*α2-antiplasmin (percent activity)+31.1*TAFI (percent activity)+1.49 PAI-1 (ug/L), and further comprising administering to the patient a suitable dose of plasminogen activating agent, optionally a reduced dose of a plasminogen activating agent, relative to the regular dose to be administered to a patient that is not determined as being at increased risk of hemorrhage, and optionally treating the patient with one or more anti-thrombotic agents that are not plasminogen activating agents, such as an agent selected from the group consisting of direct fibrinolytic agents, plasmin, delta plasmin, miniplasmin, microplasmin, lumbrokinase and nattokinase. Suitably, the patient is determined as being at increased risk of hemorrhage on administration of the plasminogen activating agent when a clot lysis time (CLT) is less than a first predetermined cutoff time.

There is also provided a plasminogen activating agent for use in a method of treating thrombosis in a patient, wherein the patient has been determined as being as suitable for treatment with the plasminogen activating agent when a clot lysis time (CLT) is equal to or greater than a first predetermined cutoff time, the method comprising determining, using at least one biomarker measurement system, a concentration of α2-antiplasmin in a blood sample of the patient, determining, using the at least one biomarker measurement system, a concentration of activated fibrinolysis inhibitor (“TAFI”) in the blood sample, and determining, using the at least one biomarker measurement system, a concentration of plasminogen activator Inhibitor 1 (“PAI-1”) in the blood sample, wherein the clot lysis time is calculated based on the concentrations of α2-antiplasmin, TAFI and PAI-1 using the equation CLT=−2,813.6+31.1*α2-antiplasmin (percent activity)+31.1*TAFI (percent activity)+1.49 PAI-1 (ug/L), and further comprising administering to the patient a dose of a plasminogen activating agent suitable to treat a thrombosis, or administering adjunctive treatments, such as increased or prolonged or repeated dosing of plasminogen activating agent. Suitably, the patient is determined as being as not at increased risk of hemorrhage on administration of the plasminogen activating agent when a clot lysis time (CLT) is equal to or greater than a first predetermined cutoff time.

While multiple embodiments are disclosed, still other embodiments of the present disclosure will become apparent to those skilled in the art from the following detailed description, which shows and describes illustrative embodiments of the invention. Accordingly, the drawings and detailed description are to be regarded as illustrative in nature and not restrictive.

It will be appreciated that numerous modifications to the abovementioned aspects of the invention may be made without departing from the scope of the invention as defined in the appended claims. Moreover, any one or more of the above described preferred embodiments could be combined with one or more of the other preferred embodiments to suit a particular application.

Optional and/or preferred features may be used in other combinations beyond those described herein, and optional and/or preferred features described in relation to one aspect of the invention may also be present in another aspect of the invention, where appropriate.

The described and illustrated embodiments are to be considered as illustrative and not restrictive in character, it being understood that only the preferred embodiments have been shown and described and that all changes and modifications that come within the scope of the invention(s) as defined in the claims are desired to be protected. It should be understood that while the use of words such as “preferable”, “preferably”, “preferred” or “more preferred” in the description suggest that a feature so described may be desirable, it may nevertheless not be necessary and embodiments lacking such a feature may be contemplated as within the scope of the invention as defined in the appended claims. In relation to the claims, it is intended that when words such as “a,” “an,” or “at least one,” are used to preface a feature there is no intention to limit the claim to only one such feature unless specifically stated to the contrary in the claim.

### BRIEF DESCRIPTION OF THE DRAWINGS

The above-mentioned and other features of this disclosure and the manner of obtaining them will become more apparent and the disclosure itself will be better understood by reference to the following description of embodiments of the present disclosure taken in conjunction with the accompanying drawings, wherein:

FIG. 1 is a chart depicting results of an experimental method for measuring clot lysis time (“CLT”) using turbidimetry;

FIG. 2 is a chart depicting results of an experimental method for measuring CLT using thromboeslastography (“TEG”);

FIG. 3 is a chart depicting a characteristic curve demonstrating normal (solid line) and resistance to fibrinolysis (dotted line) using turbidimetry;

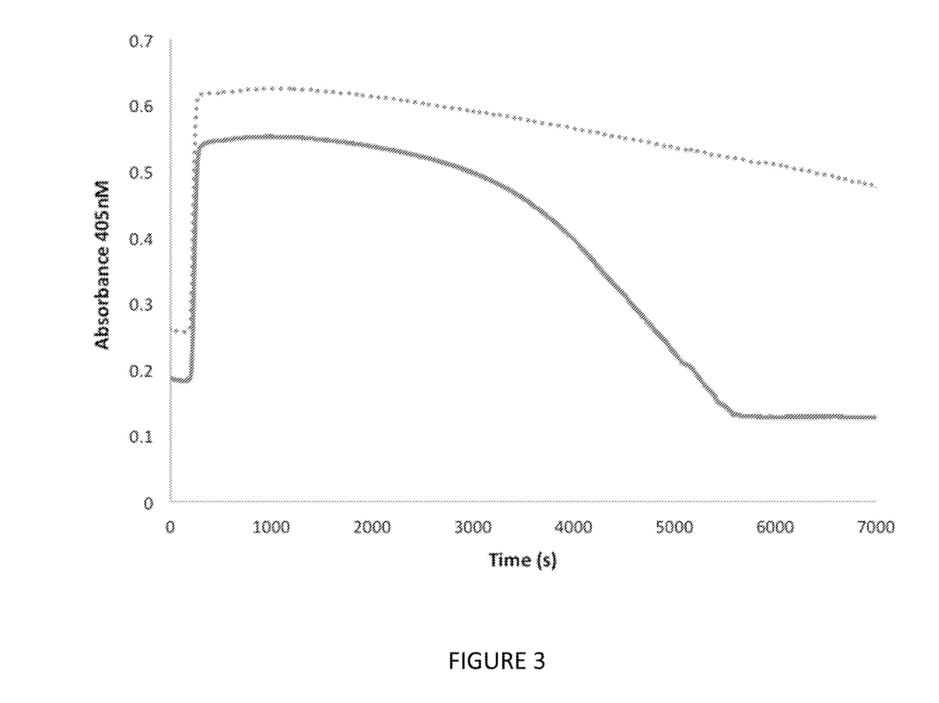

FIG. 4 is a chart depicting a characteristic curve demonstrating normal (solid line) and resistance to fibrinolysis (dotted line) using TEG;

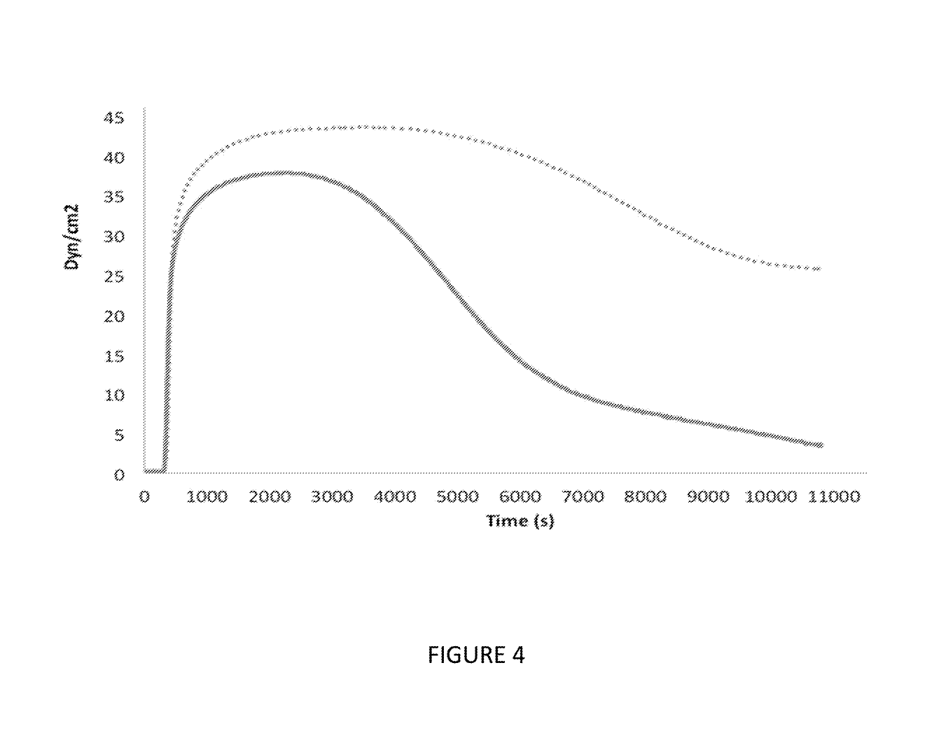

FIG. 5 is a Dot plot of CLT values for each patient in four groups;

FIG. 6 is a chart depicting a receiver operating characteristic curve using an equation according to the principles of the present disclosure; and

FIG. 7 is a block diagram of a system for carrying out the methods of the present disclosure.

While the present disclosure is amenable to various modifications and alternative forms, specific embodiments have been shown by way of example in the drawings and are described in detail below. The present disclosure, however, is not to limit the particular embodiments described. On the contrary, the present disclosure is intended to cover all modifications, equivalents, and alternatives falling within the scope of the appended claims.

### DETAILED DESCRIPTION

The following detailed description of the embodiment(s) is merely exemplary in nature and is in no way intended to limit the invention, its application, or uses.

The present disclosure is directed to a computer-based device with a predictive model executable by computer software for estimating the clot lysis time associated with a plasminogen activating agent. The computer-based device typically comprises a processor, computer hardware such as a computer screen or keyboard or disk drive, a computer software application, computer memory, data storage modules and input/output devices. The computer memory comprises instructions executable by the processor to run the predictive model of the present disclosure. The computer device may be communicatively connected to a computer network.

The predictive model of the present disclosure comprises a multivariate equation. In one embodiment, the following equation that is the basis of the predictive model where the dependent variable is clot lysis time (“CLT”), which is a surrogate marker for effectiveness of plasminogen activation on clot dissolution and determined from thromboelastography:

CLT=−2,813.6+31.1*α2-antiplasmin(percent activity)+31.1*TAFI(percent activity)+1.49 PAI-1(ug/L) Equation 1:

As a feature of the model, the multivariate equation is a linear regression equation. As another feature of the model, the equation has eight variables or factors. A high CLT measured by thromboelastography has been found to predict clinical failure of tenecteplase in patients with pulmonary embolism. A low CLT has been found to predict an increased risk of hemorrhage.

As an example of the principles of the present disclosure, work was performed using blood obtained from a prospective, multicenter trial for treatment of submassive pulmonary embolism (TOPCOAT), clinical trials identifier: NCT00680628. The methods for TOPCOAT are detailed in a separate publication (Kline J A, Hernandez-Nino J, Hogg M M et al. Rationale and methodology for a multicenter randomized trial of fibrinolysis for pulmonary embolism that includes quality of life outcomes. Emerg Med Australas. 2013(25):515-526), the entire contents of which hereby being expressly incorporated herein by reference. Patients were randomized to receive a bolus infusion of the plasminogen activator tenecteplase (TNKase®) or volume-matched 0.9% NaCl placebo. To provide negative control data, blood was used from patients who were matched to TOPCOAT patients by age and sex—plasma from apparently healthy patients who were tested for acute PE, but had no clinical evidence of PE within 90 days. To provide positive control data, plasma was used from patients with conditions known to produce hypofibrinolysis via different mechanisms, including patients with diabetes type II, and patients with metabolic syndrome (Alessi M C, Juhan-Vague I. Metabolic syndrome, haemostasis and thrombosis. Thromb Haemost. 2008; 99(6):995-1000; Dunn E J, Philippou H, Ariens R A et al. Molecular mechanisms involved in the resistance of fibrin to clot lysis by plasmin in subjects with type 2 diabetes mellitus. Diabetologia. 2006; 49(5):1071-1080). All patients in the study provided written informed consent. Blood was obtained from an arm vein, either by venipuncture or withdrawal from an indwelling venous catheter prior to treatment into a vacuum tube containing sodium citrate (BD Vacutainer®, 2.7 mL, 0.109 Molar/3.2% sodium citrate). Phlebotomy in TOPCOAT patients was performed prior to administration of study drug. Blood was immediately placed on ice, and centrifuged within 30 minutes at 4° C. at 3,000×g for 20 minutes, which has been shown to deplete platelets (Brookes, K., et al., Issues on fit-for-purpose validation of a panel of ELISAs for application as biomarkers in clinical trials of anti-Angiogenic drugs. Br J Cancer, 2010. 102(10): p. 1524-32). Plasma was immediately aliquoted and frozen at −80° C.

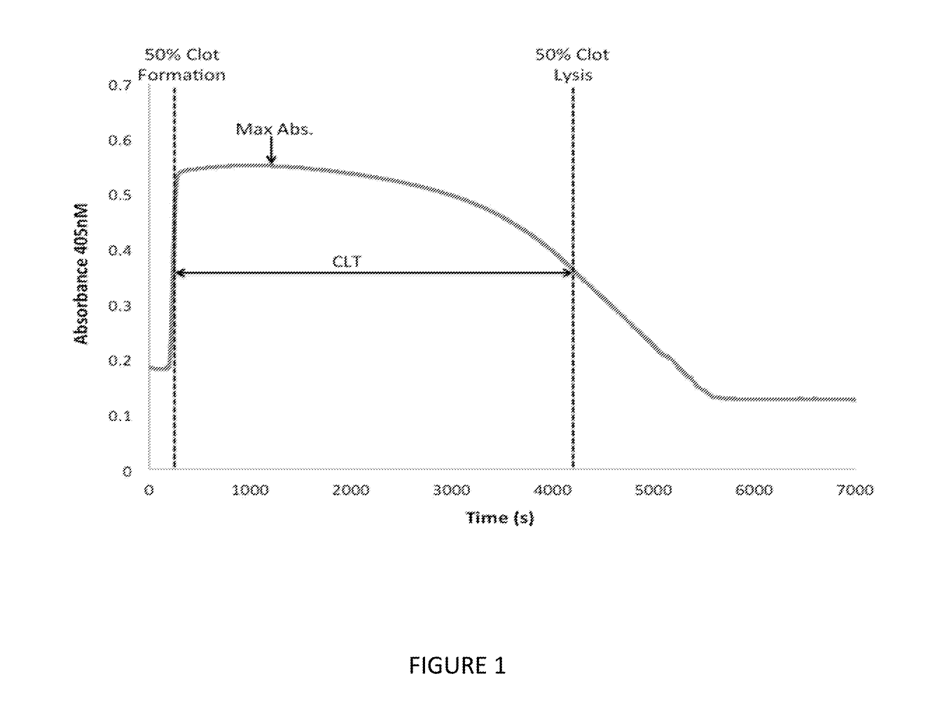

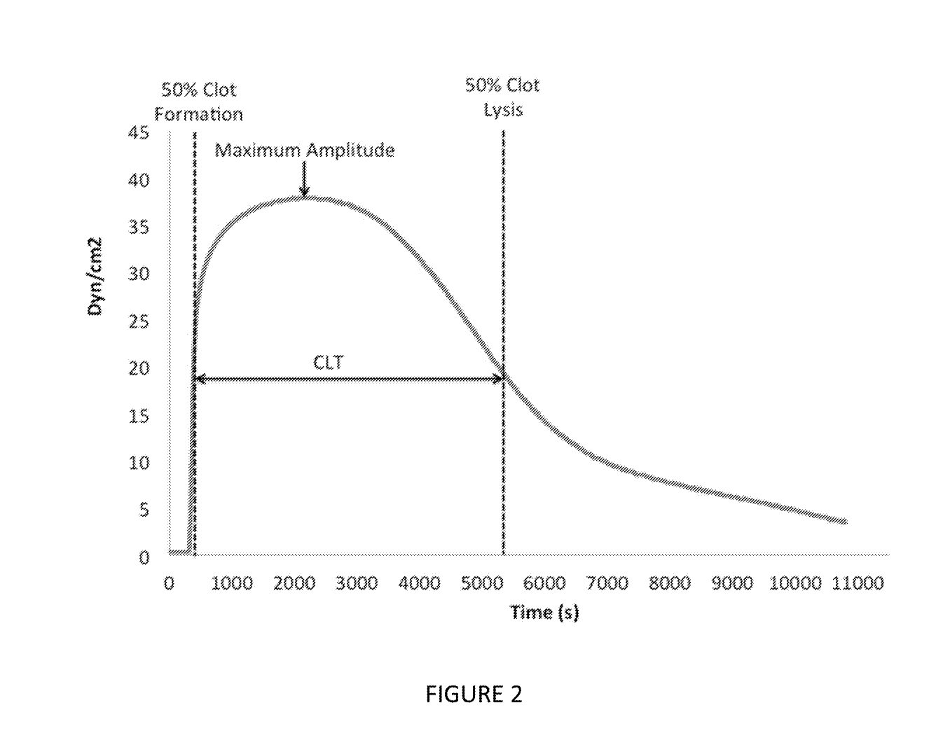

Two methods to assess CLT were used in order to mimic two distinct in-vivo physiological conditions. First, to represent CLT in stagnant (zero shear) conditions, turbidity was measured using light transmittance and a device marketed as SpectraMax by Molecular Devices of Sunnyvale, Calif. To measure CLT under shear conditions, loss of mechanical stiffness was assessed using TEG and the Haemoscope 5000 (Braintree, Mass.). For both assays, plasma comprised 50% of the total mixture volume. Turbidity was measured at 405 nm at 37° C. Coagulation was initiated by spiking plasma with calcium chloride at final concentration 15 mM, human tissue factor at a final concentration of 0.6 pM (Dade Innovin; Siemens, USA), and phospholipids at a final concentration of 12 μM (Avanti Polar Lipids; Alabaster, Al). To induce fibrinolysis, tissue plasminogen activator (“tPA”) (Alteplase, Genentech; San Francisco, Calif.) was immediately added to the plasma prior to clot formation at a final concentration of 60 ng/mL. Tris-buffered saline (50 mM Tris-HCl, 0.1 M NaCl, pH 7.4) was used as a buffering agent. Calcium chloride, tissue factor, phospholipid mixture, tPA, and the buffer were mixed in disposable TEG cups containing heparinase (Haemonetics Corporation; Braintree, Mass.) prior to the addition of plasma. Following mixing of reagents, human plasma was added to start the reaction. A 100 uL volume was transferred from the TEG cup to a 96 well plate in duplicate, and allowed to run on the spectrophotometer. The remaining reaction volume was run using TEG. Assessment of CLT by measuring turbidity was performed at an absorbance of 405 nm. The CLT is derived from a clot-lysis profile and defined as the time from the midpoint of the baseline turbidity to maximum turbidity, representing clot formation, to the midpoint of maximum turbidity back to baseline turbidity, representing the lysis of the clot (see FIGS. 1 and 2).

All plasma proteins with the exception of plasminogen activator Inhibitor 1 (“PAI-1”) were measured on the STA Compact coagulation Analyzer® (Diagnostica Stago; Parsippany N.J.) with reagents purchased from the manufacturer and analyzed as follows: Fibrinogen concentration was determined using the Clauss clotting method (STA Fibrinogen 5). α₂-antiplasmin, plasminogen and thrombin activated fibrinolysis inhibitor (“TAFI”) concentrations were determined via chromogenic assays (STA Stachrom α2-antiplasmin, STA Stachrom plasminogen and STA Stachrom TAFI). All assays were performed with the use of a commercial calibration standard; D-dimer levels were measured using a latex agglutination assay (STA Liatest D-DI). PAI-1 was quantified with a commercial ELISA assay (Life Technologies, Grand Island, N.Y.).

To assess the association of a prolonged CLT with clinical outcomes from the TOPCOAT sample, patients were grouped according to CLT value. Prolonged CLT was defined as those patients with a CLT greater than the 95 percentile from the normal control group compared with patients who had a CLT of less than or equal to the 95 percentile. The a priori outcomes assessed at three months post treatment were the results of psychometric tests for quality of life related to post-thrombotic syndrome (VEINES QoL), overall physical and mental perception of wellness from the Standard Form 36 (SF 36), as well as the SpO2(%), six minute walk distance (m), and echocardiography results.

Samples were evaluated for normality using the Shapiro-Wilk test. Means were compared using analysis of variance with Dunnett's post-hoc test to determine significance with pairwise comparisons of test groups (PE, diabetes mellitus and metabolic syndrome) versus control with P<0.05 considered significant. To determine which variables explain the change in CLT in the PE samples, a multivariate linear regression was performed with age, sex, body weight, fibrinogen, D-dimer, plasminogen, α2-antiplasmin, thrombin time, TAFI and PAI-1 as independent variables and CLT from turbidity or TEG as the dependent variable using two separate equations. The stepwise removal process was then used to select significant independent variables. Data were analyzed using SPSS (Version 22; IBM, Armonk N.Y.). Graphs were produced with Prism (Version 6.0; GraphPad, San Diego, Calif.) and SigmaPlot (version 12.0; Systat, San Jose, Calif.).

Table 1 below compares clinical features between the three control groups and the experimental group: normal (n=20), metabolic syndrome (n=10, positive control), diabetes mellitus (n=10, positive control), and intermediate risk PE (TOPCOAT). The groups were similar in age, but patients with metabolic syndrome and TOPCOAT group had a higher body mass index (p<0.05). Table 1 also compares relative concentrations and activities of biomarkers relevant to fibrinolysis in the control and PE groups. As expected, D-dimer concentrations were elevated in patients with PE. Fibrinogen levels were significantly higher in patients with PE when compared to controls. PAI-1 was significantly increased in all three test groups when compared with controls. There were no differences noted between groups in relation to a₂-antiplasmin, plasminogen, or TAFI levels.

TABLE 1
Comparison of clinical data and plasma proteins between patient groups

| | | | | TOPCOAT |
|---|---|---|---|---|
| | | Diabetes | Metabolic | (Intermediate |
| | Control | Mellitus | syndrome | risk PE) |
| Variable | (n = 20) | (n = 10) | (n = 10) | (n = 76) |
| Age | 56.5 ± 14.6 | 57.8 ± 14.2 | 65.1 ± 20 | 55 ± 13.9 |
| Male gender (%) | 11 (55%) | 5 (50%) | 7 (70%) | 46 (61%) |
| Body mass index | 27.9 ± 8.1 | 31.3 ± 5.7 | 34.7 ± 4.8* | 33.1 ± 9.1* |
| (kg/m²) | | | | |
| Diabetes Mellitus | 0 | 10 | 0 | 10 |
| Prior venous | 0 | 0 | 0 | 17 |
| thromboembolism | | | | |
| Active | 0 | 0 | 0 | 15 |
| malignancy | | | | |
| Thrombin time | 18.1 ± 2 | 19.6 ± 1.2 | 19.8 ± 1.7 | 61 ± 47.1* |
| (S) | | | | |
| Fibrinogen | 320.9 ± 54.7 | 350.7 ± 52 | 338 ± 70.7 | 412.5 ± 148.9* |
| (mg/dL) | | | | |
| α₂ antiplasmin† | 102.7 ± 12.3 | 103.2 ± 5.7 | 106.4 ± 6.7 | 99.2 ± 18.2 |
| Plasminogen† | 98.2 ± 16 | 102.3 ± 11.3 | 104.5 ± 13.7 | 109.8 ± 27.2 |
| TAFI† | 107.3 ± 16.9 | 104.3 ± 18.6 | 106.9 ± 18.7 | 99.5 ± 25.7 |
| D-dimer (μg/mL) | 0.447 ± 0.429 | 0.360 ± 0.141 | 0.429 ± 0.307 | 6.592 ± 5.102* |
| PAI-1 (pg/mL) | 1072.3 ± 780.1 | 3457 ± 2518.7* | 4171.3 ± 2177.7* | 2367.5 ± 2212.9* |

*P < 0.05 from one-way ANOVA with Dunnett's comparison with control.
** Values are listed as mean ± SD unless otherwise indicated.
*** Units are expressed as percent activity when compared to standardized controls provided by the manufacturer.
Abbreviations: TAFI—thrombolysis activated fibrinolysis inhibitor; PAI—plasminogen activator inhibitor.

Data in Table 1 were examined using pairwise comparisons of age and BMI between the three test groups versus healthy controls. With equal variances assumed, no significant difference in age was found between groups following Dunnett's post-hoc analysis. Further, no significant difference in BMI was observed between the controls metabolic syndrome group or between the controls and DM patients. There was, however, a significant difference in BMI between the control and TOPCOAT groups (p=0.041).

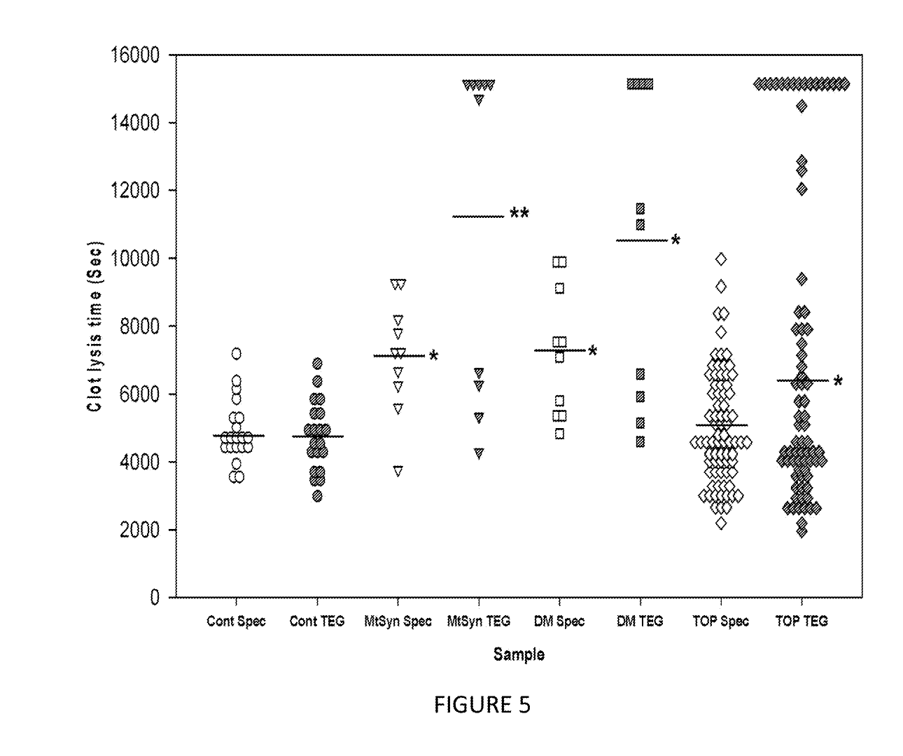

Referring now to FIG. 5, a Dot plot of CLT values for each patient in four groups is shown. Horizontal lines in the figure represent the mean of each group of data. Abbreviations used in the figure are as follows: Cont—apparently healthy control patients; TEG—thromboelastography; MtSyn—metabolic syndrome; DM—diabetes mellitus; Spec—spectrophotometry (turbidimetric method); TOP—TOPCOAT. * indicates P<0.05 vs. control, and ** indicates P<0.01 vs. control, ANOVA with Dunnett's post-hoc. The CLTs were measured with both turbidity and TEG. Using the turbidimetric technique, the mean CLT was not significantly prolonged for patients with PE compared with controls, but was prolonged in patients with diabetes mellitus and metabolic syndrome compared with controls (P=0.623, P=0.002 and P=0.003, respectively from Dunnett's). With TEG, the mean CLT was significantly prolonged for patients with PE, diabetes mellitus and metabolic syndrome compared with controls (P=0.03, P=0.0026, and P=0.0005, respectively from Dunnett's). A significantly higher proportion of patients with PE (18%) had a CLT>180 minutes compared with controls (0%) (95% confidence interval for the difference in 18%=0.3 to 27%).

To determine if a prolonged CLT has clinical significance, Table 3 below compares the mean values for the VEINES QoL score (Kahn S R, Lamping D L, Ducruet T et al. VEINES-QOL/Sym questionnaire was a reliable and valid disease-specific quality of life measure for deep venous thrombosis. Journal of Clinical Epidemiology. 2006; 59(10):1049-1056), pulse oximetry, Body mass index (kg/m2), six minute walk distance at 3 months, and the normalized mental and physical component scores (“PCS”) from the Rand Standard for (SF36) quality of life survey (Hays R D, Sherbourne C D, Mazel R M. The RAND 36-Item Health Survey 1.0. Health Econ. 1993; 2(3):217-227) in patients with and without prolonged CLT as measured by TEG. In patients given tenecteplase, significant differences were found in the VEINEs QoL score, the PCS from the SF36 between those with prolonged CLT and those without prolonged CLT. Additionally, the percentage of patients with right ventricular (“RV”) dysfunction or overload at 3 months were assessed, defined as RV dilation (>43 mm transverse diameter in diastole), RV hypokinesis, or an estimated RV systolic pressure >45 mm Hg. For those treated with tenecteplase, RV dysfunction or overload was found in 36% with prolonged CLT, versus 26% with normal CLT (95% CI for difference,−20.6 to 42.4%, exact two-sided P=0.46), and for placebo, RV dysfunction or overload was found in 54% with prolonged CLT, versus 27% with normal CLT (95% CI for difference of 17%-5.1 to 55.4%, exact two-sided P=0.095).

TABLE 3

| | VEINES QoL | | Distance | Mental Health | Physical |
|---|---|---|---|---|---|
| | score for post- | | walked in | summary | component |
| | thrombotic | Baseline | six | score from | score from |
| | syndrome | SpO2 (%) | minutes | SF36 | SF36 |
| mean Tenec prolonged CLT | 85.9 | 96.2 | 328.5 | 55.2 | 42.5 |
| SD Tenec prolonged CLT | 12.7 | 1.7 | 104.9 | 11.3 | 10.7 |
| mean Tenec normal CLT | 96.0 | 97.0 | 445.7 | 52.8 | 49.2 |
| SD Tenec normal CLT | 10.5 | 1.5 | 79.1 | 6.9 | 8.2 |
| P from unpaired t-test | 0.021 | 0.191 | 0.002 | 0.453 | 0.051 |
| mean Placebo prolonged CLT | 93.5 | 97.0 | 411.6 | 54.3 | 41.5 |
| SD Placebo prolonged CLT | 14.4 | 1.3 | 97.0 | 8.8 | 14.1 |
| mean Placebo normal CLT | 87.7 | 97.0 | 399.4 | 51.4 | 41.7 |
| SD Placebo normal CLT | 18.7 | 1.6 | 122.6 | 13.9 | 12.8 |
| P from unpaired t-test | 0.326 | 0.934 | 0.766 | 0.494 | 0.969 |

Taken together, data from FIG. 5 indicate the predictivenss of the CLT from TEG. The study then sought to determine predictors of CLT in seconds from TEG in PE patients using multivariate linear regression for each technique. The model included factors that are directly or indirectly known to affect probability of response to lysis and risk of hemorrhage, including age, body mass index (“BMI”), fibrinogen (“FIB”), D-dimer concentration, plasminogen, (“PLG”), α2 antiplasmin (“AP”), thrombin time (“TT”), and thrombin activated thrombolysis inhibitor (“TAFI”), and plasminogen activator inhibitor 1 (“PAI-1”) concentrations. Table 4 below shows the results of a regression analysis done on the CLT data for 76 patients in the original TOPCOAT dataset (Kline J A, Kabrhel C., Courtney D M et al. Treatment of submassive pulmonary embolism with tenecteplase or placebo: cardiopulmonary outcomes at three months (TOPCOAT): Multicenter double-blind, placebo-controlled randomized trial. J Thromb & Haemost. 2014). The analysis was performed with the statistical program StatsDirect (v 10.131). The values denoted by b0 . . . b9 represent the beta coefficients in the equation and the t and P values are the significance tests for each coefficient.

TABLE 4

| | | | | |
|---|---|---|---|---|
| Intercept | b0 = −4,540.769 | | t = −1.179 | P = 0.243 |
| Age | b1 = 14.34 | r = 0.059 | t = 0.482 | P = 0.631 |
| BMI | b2 = 34.766 | r = 0.095 | t = 0.777 | P = 0.44 |
| FIB | b3 = −0.054 | r = −0.002 | t = −0.015 | P = 0.988 |
| D-Dimer | b4 = 27.739 | r = 0.041 | t = 0.332 | P = 0.741 |
| PLG | b5 = −19.227 | r = −0.102 | t = −0.832 | P = 0.409 |
| AP | b6 = 47.24 | r = 0.21 | t = 1.741 | P = 0.086 |
| TT | b7 = −7.076 | r = −0.099 | t = −0.808 | P = 0.422 |
| TAFI avg (calc) | b8 = 41.343 | r = 0.24 | t = 2.007 | P = 0.049 |
| PAI-1 (correct | b9 = 1.478 | r = 0.683 | t = 7.603 | P < 0.001 |
| analysis) | | | | |

TEG CLT = −4,540.769 +14.34 Age +34.766 BMI −0.054 FIB +27.739 D-Dimer −19.227 PLG +47.24 AP −7.076 TT +41.343 TAFI avg (calc) +1.478 PAI-1 (correct analysis).

Next an analysis of variance from regression was performed as follows:

| Source of variation | Sum Squares | DF | Mean Square |
|---|---|---|---|
| Regression | 9.351509E+008 | 9 | 1.039057E+008 |
| Residual | 7.462891E+008 | 66 | 11,307,409.921 |
| Total (corrected) | 1.681440E+009 | 75 | |

Root MSE = 3,362.649
F = 9.189 P < 0.001
Multiple correlation coefficient (R) = 0.746
R² = 55.616%
Ra² = 49.564%
Durbin-Watson test statistic = 1.372

### Multiple Regression—Best Subset

### Selected Variables:

AP

TAFI avg (calc)

PAI-1 (correct analysis)

F=28.225

R²=0.54

Mallows' Cp=2.336

### Multiple Linear Regression

| | | | | |
|---|---|---|---|---|
| Intercept | b0 = −2,813.581 | | t = −1.228 | P = 0.223 |
| AP | b1 = 35.346 | r = 0.185 | t = 1.601 | P = 0.114 |
| TAFI avg (calc) | b2 = 31.066 | r = 0.228 | t = 1.985 | P = 0.051 |
| PAI-1 (correct | b3 = 1.494 | r = 0.714 | t = 8.653 | P < 0.001 |
| analysis) | | | | |

TEG CLT = −2,813.581 +35.346 AP +31.066 TAFI avg (calc) +1.494 PAI-1 (correct analysis).

### Multiple Linear Regression—Prediction

TEG CLT=7,318.816 (least squares mean)

95% Confidence interval=6,569.712 to 8,067 92

95% Prediction interval=745.453 to 13,892.178

CLT from thromboelastography=−2,813.6+31.1*α2-antiplasmin(percent activity)+31.1*TAFI(percent activity)+1.49 PAI-1(ug/L). Equation (1):

This equation thus uses the measurement of three plasma proteins to estimate the CLT from thromboelastography, which was previously shown to predict clinically important outcomes. The following data provide descriptive statistical values for the result of Equation 1 in the TOPCOAT population.

| | | Tenecteplase | Placebo |
|---|---|---|---|
| Variables | All | treated | treated |
| Valid data | 76 | 36 | 40 |
| Missing data | 7 | 3 | 3 |
| Sum | 543,143.09 | 236,929.614 | 304,103.997 |
| Mean | 7,146.62 | 6,581.378 | 7,602.6 |
| Variance | 11,818,451.154 | 7,589,679.392 | 14,784,305.412 |
| Standard deviation | 3,437.797 | 2,754.937 | 3,845.036 |
| Variance coefficient | 0.481 | 0.419 | 0.506 |
| Standard error of mean | 394.342 | 459.156 | 607.954 |
| Upper 95% CL of | 7,932.19 | 7,513.515 | 8,832.302 |
| mean | | | |
| Lower 95% CL of | 6,361.049 | 5,649.241 | 6,372.898 |
| mean | | | |
| Geometric mean | * | * | * |
| Skewness | 2.009 | 2.072 | 1.996 |
| Kurtosis | 8.429 | 7.521 | 7.933 |
| Maximum | 23,043.417 | 16,995.795 | 23,043.417 |
| Upper quartile | 8,456.127 | 7,554.542 | 9,456.283 |
| Median | 6,552.58 | 6,034.587 | 7,308.727 |
| Lower quartile | 4,921.705 | 4,911.542 | 5,335.679 |
| Interquartile range | 3,534.421 | 2,642.999 | 4,120.604 |
| Minimum | 2,109.48 | 2,109.48 | 3,253.541 |
| Range | 20,933.938 | 14,886.316 | 19,789.876 |
| Centile 95 | 15,247.843 | 16,995.795 | 19,145.63 |
| Centile 5 | 3,923.352 | 4,165.33 | 3,958.125 |

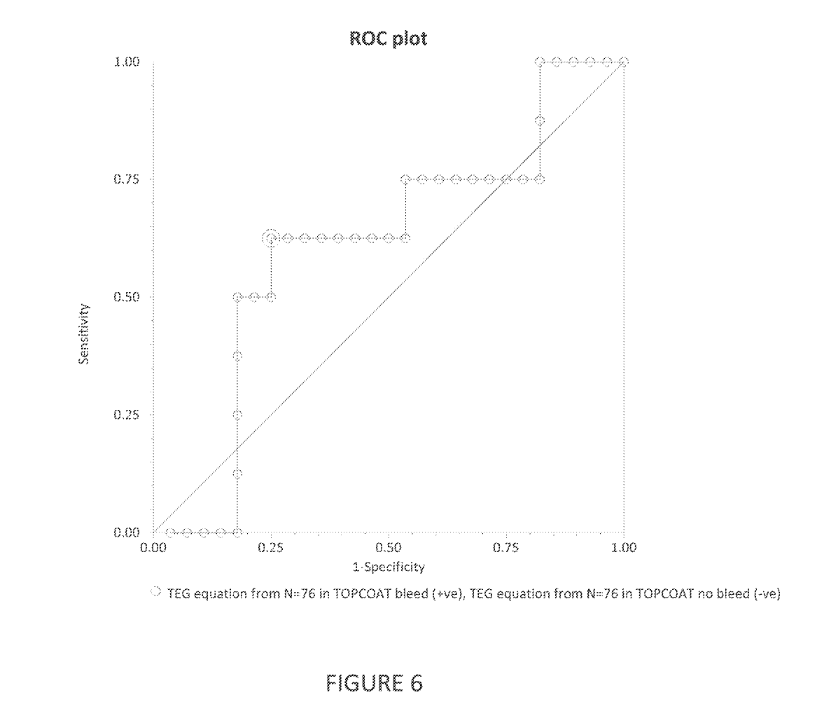

FIG. 6 shows the performance of Equation 1 in terms of its ability to predict patients who had a major or clinically relevant non-major bleed and were treated with tenecteplase. FIG. 6 is a receiver operating characteristic curve using the result of Equation 1 to predict the outcome of hemorrhage that was observed in 8 of 36 patients treated with the plasminogen activator tenecteplase from the TOPCOAT population. To produce the ROC properly, the actual value of the equation was subtracted from the maximal value that was found in the entire TOPCOAT dataset (namely 23,041 seconds). At a cutoff of area under ROC curve by extended trapezoidal rule=0.678309 (result obtained by 23,041-estimated CLT from Equation 1). The Wilcoxon estimate of area under ROC curve=0.607 (95% CI=0.373 to 0.841). The optimum cut-off point selected=18,115 seconds, but because this value must be subtracted from 23,041, the optimal cutoff is 4,926 seconds, which corresponds closely to the mean CLT from the study group without hemorrhage. At values above this number, three patients had clinically relevant but non-major hemorrhage (5/7 or 62.5% sensitivity for detection of those at risk of bleeding) and this included 75% of patients without bleeding (75% specificity). Patients with values below this number are at increased risk of hemorrhage from standard dose plasminogen activator agents. All eight patients who had major or clinically relevant non-major bleeding and who were treated with the standard dose of the plasminogen activator tenecteplase had a TEG CLT time <=4,926 seconds (100% sensitivity) and 19/28 patients who were treated with standard dose tenecteplase had a TEG CLT time <=4,926 seconds, meaning that 11/28 had a value >4,926 seconds (39% specificity).

From the 36 patients treated with tenecteplase the study then examined the significance of a TEG CLT<4,926 seconds, estimated from Equation 1 to predict a bleeding outcome that could be related to tenecteplase administration to humans. To test for the significance of the TEG CLT>4,926 seconds from Equation 1, a standard odds ratio was performed, represented below.

| | |
|---|---|
| 5 | 3 |
| 7 | 21 |

Observed odds ratio = 5
Conditional maximum likelihood estimate of odds ratio = 4.744
Exact Fisher 95% confidence interval = 0.714 to 38.952
Exact Fisher one sided P = 0.062, two sided P = 0.086
Exact mid-P 95% confidence interval = 0.877 to 29.863
Exact mid-P one sided P = 0.036, two sided P = 0.071

From the 36 patients treated with tenecteplase the study then examined the significance of a TEG CLT>4,926 seconds, estimated from Equation 1 to predict an adverse outcome that could be related to inadequate clot lysis in the human. These adverse outcomes, assessed at three months, included a low six minute walk distance (<330 m), or a PCS from the SF36<30 points, or right ventricular dilation or hypokinesis or estimated right ventricular systolic pressure >45 mm Hg on echocardiography. To test for the significance of the TEG CLT>4,926 seconds from Equation 1, a standard odds ratio was performed, represented below. This analysis excludes the eight patients with a hemorrhagic outcome, and therefore only includes 28 patients.

### Exact Confidence Limits for 2 by 2 Odds

### Input Table:

| | |
|---|---|
| 12 | 3 |
| 9 | 4 |

Observed odds ratio = 1.778
Conditional maximum likelihood estimate of odds ratio = 1.741
Exact Fisher 95% confidence interval = 0.229 to 15.062
Exact Fisher one sided P = 0.412, two sided P = 0.67
Exact mid-P 95% confidence interval = 0.289 to 11.505
Exact mid-P one sided P = 0.275, two sided P = 0.549

Thus, a TEG CLT>4,926 seconds, as estimated from Equation 1 had a slight tendancy to predict a worsened outcome in terms of exercise tolerance, quality of life or echocardiographic finding at 3 months for patients treated with tenecteplase. This suggests that patients with a value >4,926 are less likely to benefit from standard dose plasminogen activator treatment delivered by systemic infusion.

As should be apparent to those skilled in the art, patients with TEG CLT value at or below 4,926 seconds estimated from Equation 1 could benefit from reduced dose plasminogen activator fibrinolytic treatment, whether delivered systemically or with a catheter positioned in close proximity to the thrombus. Moreover, it should also be apparent that patients with a TEG CLT over 4,926 seconds estimated from Equation 1 may benefit from adjunctive treatments including increased or prolonged or repeated dosing of plasminogen activator agent, delivered either systemically by catheter immediately proximal to the thrombus. The finding of a prolonged value from Equation 1 also indicates the need to use of a device that imparts mechanical, ultrasonic or other method of energy transfer to enhance fibrinolysis.

The finding of more extreme TEG CLT values from Equation 1 could indicate the need for alternative treatment to plasminogen activators. Patients with a value below the 5 percentile (3,923 seconds) could be considered at very high risk of hemorrhage and patients with values above the 95th percentile (15,247 seconds) could be considered at very high risk of clinical failure with treatment with plasminogen activating agents. Therefore, these patients should be considered for treatment with alternative agents, including the so-called direct fibrinolytic agents, plasmin, delta plasmin, miniplasmin or microplasmin, or the use of alternative fibrinolytic agents lumbrokinase or nattokinase, or the use of surgical embolectomy or the use of purely mechanical means of clot removal.

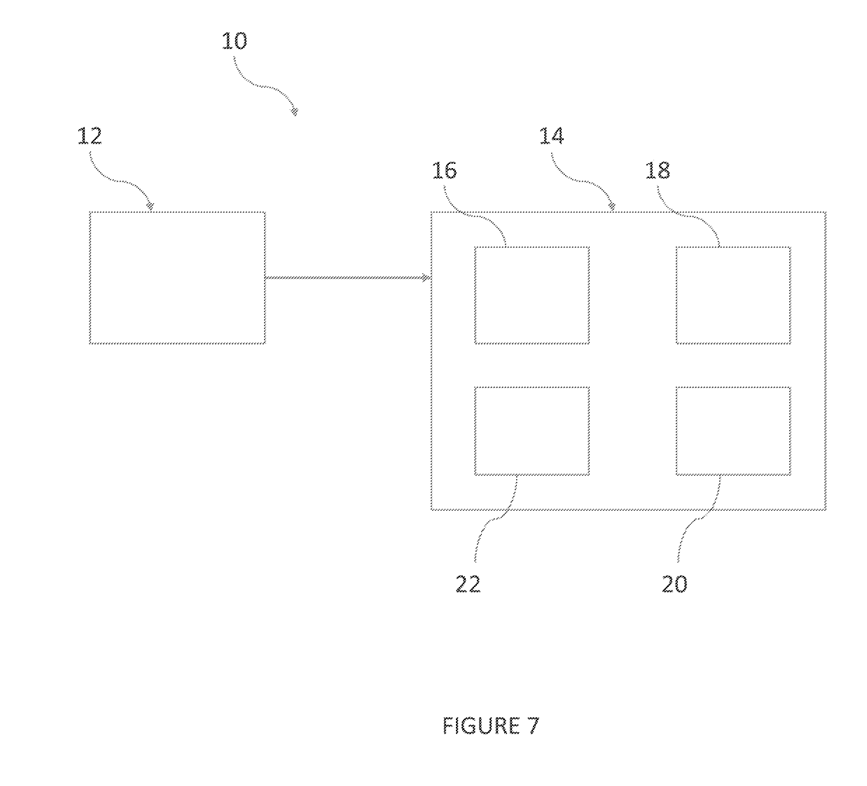

Referring now to FIG. 7, a system is depicted for carrying out the above-described principles of the present disclosure. System 10 generally includes a biomarker concentration measurement system 12 and a computing device 14. Biomarker concentration measurement system 12 may include a plurality of different hardware and software components. For example, as described above, system 12 may be configured to measure a concentration of α2-antiplasmin in a blood sample of the patient, a concentration of activated fibrinolysis inhibitor (“TAFI”) in the blood sample, and a concentration of plasminogen activator Inhibitor 1 (“PAI-1”) in the blood sample. System 12 may include an STA Compact coagulation Analyzer® (Diagnostica Stago; Parsippany N.J.) with reagents purchased from the manufacturer and analyzed as follows: Fibrinogen concentration was determined using the Clauss clotting method (STA Fibrinogen 5). System 12 may include a chromogenic assay with a commercial calibration standard for determining a concentration α2-antiplasmin, plasminogen and thrombin activated fibrinolysis inhibitor (TAFI). System 12 may further include a commercial ELISA assay (Life Technologies, Grand Island, N.Y.) for determining a plasminogen activator Inhibitor 1 (“PAI-1”) concentration within a sample of the patient's blood. Any and all of these components (and the associated software) are represented by system 12.

Computing device 14 generally includes an interface 16 which receives data from system 12, a processor 18, a memory 20 and a user interface 22. Computing device 14 may receive data representing biomarker concentrations from system 12 through a wired or wireless connection. While computing device 14 is depicted as including a single processor 18, it should be understood that multiple processors may be used, either as a part of computing device 14 or part of a distributed network of processors. Memory 20 may include non-transient instructions for execution by processor 18 to perform the functions described above, including but not limited to carrying out the computation of CLT for the blood sample and its comparison to the various predetermined cutoff times for predicting or estimating clinical responsiveness of a patient to administration of a plasminogen activating agent as described herein. Memory 20 may also include the predetermined cutoff times and other parameters necessary for performing the various calculations described herein. While memory 20 is depicted as a single component, it should be understood that multiple memory devices may be incorporated (or associated with) computing device 14 according to the principles of the present disclosure. User interface 22 is generically depicted as a single device, but it should be understood that user interface 22 may include a plurality of different devices (and associated software) for receiving user input and providing output to the user of computing device 14, including but not limited to a display, keyboard, mouse, touch-screen, alarm, or audio/visual communication device, which either directly receives and provides information to/from the user or does so indirectly through other intervening devices.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A method for estimating the clinical responsiveness of a patient to a dose of a plasminogen activating agent to treat a thrombosis, comprising:
determining, using at least one biomarker measurement system, a concentration of α2-antiplasmin in a blood sample of the patient;
determining, using the at least one biomarker measurement system, a concentration of activated fibrinolysis inhibitor (“TAFI”) in the blood sample;
determining, using the at least one biomarker measurement system, a concentration of plasminogen activator Inhibitor 1 (“PAI-1”) in the blood sample;
computing, using a computing device, a clot lysis time (“CLT”) based on the concentrations of α2-antiplasmin, TAFI and PAI-1 using the equation CLT=−2,813.6+31.1*α2-antiplasmin (percent activity)+31.1*TAFI (percent activity)+1.49 PAI-1 (ug/L); and
determining, using the computing device, that the patient is at increased risk of hemorrhage when the computed CLT is less than a first predetermined cutoff time.

2. The method of claim 1 wherein the first predetermined cutoff time is approximately 4,926 seconds.

3. The method of claim 1 further including determining, using the computing device, that the patient is at an increased risk of clinical failure with treatment with the plasminogen activating agent when the computed CLT is greater than a second predetermined cutoff time, the second predetermined cutoff time being greater than the first predetermined cutoff time.

4. The method of claim 3 wherein the second predetermined cutoff time is approximately 15,247 seconds.

5. The method of claim 1 wherein the concentrations of α2-antiplasmin and TAFI are percentages of a normative value.

6. The method of claim 1 wherein the at least one biomarker measurement system includes a chromogenic assay for determining the concentration of α2-antiplasmin, TAFI and PAI-1 in the blood sample.

7. The method of claim 1 further including responding to the computed CLT being less than the first predetermined cutoff time by providing a reduced dose plasminogen activator fibrinolytic treatment to the patient.

8. A system for estimating the clinical responsiveness of a patient to administration of a plasminogen activating agent to treat a thrombosis, comprising:
at least one biomarker measurement system for determining a concentration of α2-antiplasmin in a blood sample of the patient, a concentration of activated fibrinolysis inhibitor (“TAFI”) in the blood sample, and a concentration of plasminogen activator Inhibitor 1 (“PAI-1”) in the blood sample;
a computing device in communication with the at least one biomarker measurement system including a processor and a memory including instructions which when executed by the processor cause the computing device to compute a clot lysis time (“CLT”) based on the concentrations of α2-antiplasmin, TAFI and PAI-1 using the equation CLT=−2,813.6+31.1*α2-antiplasmin (percent activity)+31.1*TAFI (percent activity)+1.49 PAI-1 (ug/L), and to compare the computed CLT to a first predetermined cutoff time; and
a user interface configured to provide a user of the computing device information that the patient is at increased risk of hemorrhage when the computed CLT is less than a first predetermined cutoff time.

9. The system of claim 8 wherein the first predetermined cutoff time is approximately 4,926 seconds.

10. The system of claim 8 wherein the user interface is further configured to provide the user information that the patient is at an increased risk of clinical failure with treatment with the plasminogen activating agent when the computed CLT is greater than a second predetermined cutoff time, the second predetermined cutoff time being greater than the first predetermined cutoff time.

11. The system of claim 10 wherein the second predetermined cutoff time is approximately 15,247 seconds.

12. The system of claim 8 wherein the concentrations of α2-antiplasmin and TAFI are percentages of a normative value.

13. The system of claim 8 wherein the at least one biomarker measurement system includes a chromogenic assay for determining the concentration of α2-antiplasmin, TAFI and PAI-1 in the blood sample.
